(19) United States
(12) Patent Application Publication
(10) Pub. No.: US 2026/0034572 A1
(43) Pub. Date: Feb. 5, 2026

(54) TEXTILE WASTE RECYCLING SYSTEMS AND METHODS

(71) Applicant: 14787285 Canada Inc. dba Nuria Scientific, London (CA)

(72) Inventors: Nicholas LEROY LEDRA, London (CA); Wankei WAN, London (CA)

(21) Appl. No.: 18/790,690

(22) Filed: Jul. 31, 2024

(51) Int. Cl. B09B 3/60; B09B 3/35; B09B 3/80; B09B 101/85; C08J 11/08; C12M 1/40

(52) U.S. Cl. CPC B09B 3/60; B09B 3/35; B09B 3/80; C08J 11/08; C12M 21/18; B09B 2101/85; C08J 2301/02; C08J 2367/00

(57) ABSTRACT

Present disclosure provides a system to process textile waste. Said system comprises a dissolution chamber that receives the textile waste, wherein said dissolution chamber stores a first organic solvent to dissolve a polyester fraction of the received textile material, producing a suspension of undissolved cotton in said first organic solvent. A first filtering unit receives said suspension and generates a polyester liquid stream and a solid cotton fraction. A precipitation unit receives said polyester liquid stream and a second organic solvent to generate a precipitated polyester dispersion. A second filter separates solid polyester from a cocktail based on said first and second organic solvents. A first solvent recovery unit separates said organic solvents, returning said first organic solvent to said dissolution chamber and said second organic solvent to said precipitation unit. A bioreactor enables enzymatic treatment of said solid cotton fraction.

### TECHNICAL FIELD

The present disclosure generally relates to recycling. Further, the present disclosure particularly relates to systems and methods of processing textile waste.

### BACKGROUND

Textile waste management has become a significant concern due to the increasing volume of discarded textile materials. Traditional disposal methods, such as landfilling and incineration, present substantial environmental challenges, including the release of harmful pollutants and the depletion of landfill space. Recycling and recovery of textile materials offer a more sustainable alternative, contributing to the conservation of resources and reduction of environmental impact. The recycling of polyester and cotton, two predominant components in textile waste, poses particular challenges due to the different chemical properties of these materials. Additionally, the fast fashion trends in the textile industry have resulted in high consumption of fiber with concomitant generation of waste. Awareness of environmental pollution resulting from textile production and disposal has increased significantly. This increase has pushed toward more sustainable recycling alternatives to properly handle the end-of-life of textiles. The textile industry generates significant amounts of waste, including large quantities of polyester and cotton materials. Efficiently managing such waste remains a critical challenge.

Various methods have been developed to address textile waste, particularly focusing on recycling and reusing fibers to minimize environmental impact. Mechanical recycling involves shredding and reprocessing textile fibers. The mechanical recycling method is straightforward and widely used; however, often results in degraded fiber quality and limited applicability for high-quality textile production. Moreover, mechanical recycling does not effectively separate polyester and cotton fibers, leading to mixed fiber outputs that are difficult to utilize in new textile products. Specifically, the recovery of cotton fiber through mechanical recycling is problematic, as the shredding process shortens the cotton fibers, reducing their spinnability and strength, which are critical for yarn production. Consequently, the resulting cotton fibers are of inferior quality, limiting their use in producing durable and high-quality yarns, threads and clothing.

Thermal recycling techniques utilize heat to process textile waste. These methods involve melting synthetic fibers such as polyester while leaving natural fibers like cotton intact. Subsequent mechanical separation segregates the melted polyester from the solid cotton. While thermal recycling can effectively separate polyester from other materials, it suffers from high energy consumption and potential thermal degradation of fibers. Furthermore, the handling and processing of melted polyester present significant operational challenges. For cotton fibers, thermal methods are less effective because they do not address the physical properties required for yarn production. The thermal processes can cause weakening or partial degradation of cotton fibers, further complicating their reuse in high-quality textile applications.

In addition to the above-mentioned methods, hybrid approaches combining mechanical and thermal have been explored. While such methods potentially enhance recycling efficiency, they also introduce complexity in process control and integration. Managing multiple treatment steps, solvent recovery and fiber quality remains a substantial challenge. These hybrid methods aim to leverage the advantages of different recycling techniques, but they also amplify their respective limitations. For instance, combining mechanical and chemical methods can exacerbate the fiber degradation issues inherent in both processes, leading to further deterioration of cotton fiber quality. As a result, the recycled cotton fibers often fail to meet the standards required for producing high-strength, high-quality yarns.

Moreover, the handling of cotton and polyester blends presents specific challenges. The intertwined nature of cotton and polyester fibers in blended fabrics complicates their separation. Mechanical methods struggle to isolate these fibers effectively, resulting in mixed fiber outputs that are of limited use. Thermal methods face similar issues, as the melting point of polyester is much lower than the degradation point of cotton, leading to difficulties in achieving complete separation without damaging the cotton fibers. Chemical methods, while capable of dissolving polyester, often fail to preserve the cotton fibers in a condition suitable for high-quality applications. The solvents used can degrade the cotton fibers, affecting their length and strength, which are critical for yarn production.

In light of the above discussion, there exists an urgent need for solutions that overcome the problems associated with conventional systems and/or techniques for efficiently separating and recycling polyester and cotton fibers from textile waste while optimizing recovery and reuse of textile components.

### SUMMARY

In an aspect, the present disclosure provides a system for processing of textile waste. The system comprises a dissolution chamber that receives the textile waste. The dissolution chamber stores a first organic solvent that dissolves a polyester fraction of the received textile material to produce a suspension of undissolved cotton fiber in the first organic solvent. The system further comprises a first filtering unit receives the suspension of undissolved cotton in the first organic solvent and generates a polyester liquid stream and a solid cotton fraction. Moreover, the system comprises a precipitation unit that receives the polyester liquid stream and a second organic solvent to generate a precipitated polyester dispersion. The system also comprises a second filter that receives the precipitated polyester dispersion to separate a solid polyester from a cocktail based on the first organic solvent and the second organic solvent. Additionally, the system comprises a first solvent recovery unit that receives the cocktail to separate the first organic solvent and the second organic solvent. The first organic solvent is transferred to the dissolution chamber and the second organic is transferred to the precipitation unit. The system further comprises a bioreactor that receives the solid cotton fraction. The bioreactor enables an enzymatic treatment of the received cotton in a treatment medium comprising at least one enzyme, water and buffer at a controlled temperature between 15° C. and 60° C., at a pre-set pressure ranging from 50 to 80 kPa, an enzyme concentration for a pre-set time period from 15 minutes to 30 days to generate a colloidal solution of the cotton fibers. Moreover, the system comprises a third filter that receives the colloidal solution of the cotton fibers and separates the cotton fibers and a liquid fraction comprises the enzyme and sugar. Also, the system comprises a sugar recovery unit that receives the liquid fraction from the second filter and removes the sugar from the liquid fraction to produce the treatment medium that is transferred to the bioreactor.

In an embodiment, each of the first filtering unit and the second filter unit comprises multiple motor-driven brushes to sweep across a filter surface to dislodge the accumulated particles.

In another embodiment, each of first filtering unit and second filter unit comprises a plurality of vibrators or sonicator attached to an exterior of the filter housing. Further, each vibrator generates vibration at specific intervals to maintain a filtration flow rate.

In yet another embodiment, the bioreactor comprises the immobilized enzyme beads.

In still another embodiment, the precipitation unit comprises a double-walled chamber to receive a circulating fluid within the walls to maintain a thermal parameter during precipitation of the polyester.

In a further embodiment, the sugar recovery unit comprises a sugar affinity column to selectively bind sugar while allowing the enzyme to pass therethrough.

In a further embodiment, the second filter further comprises a centrifuge mechanism to separate the solid polyester from the cocktail based on the second organic solvent.

In a further embodiment, the bioreactor comprises an automated pH control unit to maintain an optimal pH conditions during the enzymatic treatment.

In a further embodiment, the precipitation unit comprises the baffles to facilitate separation of the precipitated polyester.

In a further embodiment, the bioreactor comprises a temperature-controlled jacket to maintain a controlled temperature throughout the enzymatic treatment.

In a further embodiment, the sugar recovery unit comprises a membrane filtration system to concentrate the treatment medium, before recycling to the bioreactor.

In an embodiment, the system further comprises a shredder unit positioned at an upstream of the dissolution chamber. The shredder unit comprises a conveyor belt to uniformly feed the textile waste to the blades to shred the feed waste textile into the textile fragments and a dust extraction unit to capture and remove the airborne particles.

In an embodiment, the system further comprises a bleaching unit-better to say dye removal unit-removes brighteners-finishing treatments-non-wrinkle-positioned upstream of the dissolution chamber. The bleaching unit comprises chemical spray nozzles to apply a bleaching agent to the textile waste to remove a dye and a mixing chamber to enable application of the bleaching agent-ozones, dye removing enzymes on the textile waste.

In another aspect, the present disclosure provides a method of processing textile waste. The method comprises receiving the textile waste in a dissolution chamber. The dissolution chamber stores a first organic solvent that dissolves a polyester fraction of the received textile material to produce a suspension of undissolved cotton in the first organic solvent, receiving the suspension of undissolved cotton in the first organic solvent into a first filtering unit and generating a polyester liquid stream and a solid cotton fraction, receiving the polyester liquid stream and a second organic solvent in a precipitation unit to generate a precipitated polyester dispersion, receiving the precipitated polyester dispersion in a second filter to separate a solid polyester from a cocktail based on the first organic solvent and the second organic solvent and receiving the cocktail in a first solvent recovery unit to separate the first organic solvent and the second organic solvent. The first organic solvent is transferred to the dissolution chamber and the second organic solvent is transferred to the precipitation unit. The method further comprises receiving the solid cotton fraction in a bioreactor. The bioreactor enables an enzymatic treatment of the received cotton in a treatment medium comprising at least one enzyme, water and buffer at a controlled temperature between 15° C. and 60° C., at a pre-set pressure ranging from 50 to 80 kPa, an enzyme concentration for a pre-set time period from 15 minutes to 30 days to generate a colloidal solution of the cotton fibers. Moreover, the method comprises receiving the colloidal solution of the cotton fibers in a third filter and separating the cotton fibers and a liquid fraction comprising the enzyme and sugar and receiving the liquid fraction from the second filter in a sugar recovery unit and removing the sugar from the liquid fraction to produce an enzyme solution that is transferred to the bioreactor.

In an embodiment, the method comprises receiving a circulating fluid within a precipitation unit comprising a double-walled chamber to maintain a thermal parameter during precipitation of the polyester.

In another embodiment, the method comprises separating the solid polyester from the cocktail based on the second organic solvent in a centrifuge mechanism.

In yet another embodiment, the method comprises maintaining optimal pH conditions during the enzymatic treatment using automated pH control unit and the bioreactor comprises the automated pH control unit.

In still another embodiment, the method comprises maintaining a controlled temperature throughout the enzymatic treatment using a temperature-controlled jacket and the bioreactor comprises the temperature-controlled jacket.

In a further embodiment, the method comprises concentrating treatment medium using a membrane filtration system, before recycling to the bioreactor and the sugar recovery unit comprises the membrane filtration system.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein.

Embodiments of the present disclosure will now be described, by way of example only, with reference to the following diagrams.

Referring to FIG. 1, there is shown a block diagram of a system 100 to process textile waste 102, according to an embodiment of the present disclosure;

FIG. 2, there is shown a flow diagram illustrating processing of cotton fibers subsequent to passage through the third filter, in accordance with an embodiment of the present disclosure;

FIG. 3, there is shown a flow diagram illustrating processing of solid polyester subsequent to passage through the second filter, in accordance with an embodiment of the present disclosure;

FIG. 4, there is schematically illustrated presence of an additional step in the processing of textile waste, in accordance with an embodiment of the present disclosure; and

FIG. 5, there is shown a flowchart 500 illustrating a method of processing textile waste, in accordance with an embodiment of the present disclosure.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

As used herein, the term “system” refers to an arrangement of interconnected components designed to process textile waste. The purpose of the system of present disclosure is to efficiently separate and recycle various fractions of textile materials through a series of chemical, biochemical and mechanical treatments. The system incorporates multiple units such as a dissolution chamber, filtering units, precipitation unit, bioreactor and recovery units, each performing specific functions for complete processing of textile waste. Under exemplary operating conditions, the system can handle large quantities of textile waste, optimizing resource recovery and minimizing environmental impact and also enable sustainable waste management practices in the textile industry.

As used herein, the term “textile waste” refers to discarded or leftover fabric materials from textile production or post-consumer use. The textile waste undergoes various processing steps to recover valuable components like cotton and polyester. Textile waste can include a variety of fabrics, such as blends of natural and synthetic fibers. Processing textile waste helps in reducing landfill accumulation and recovering reusable materials, thereby promoting environmental sustainability.

As used herein, the term “dissolution chamber” refers to a vessel designed to receive and store textile waste along with an organic solvent. The dissolution chamber enables dissolution of polyester fraction of the textile material. The dissolution chamber utilizes a stirrer/agitator for adequate mixing of organic solvent with the textile waste to facilitate the dissolution process. Under exemplary operating conditions, the dissolution chamber maintains specific temperature and agitation settings to optimize the dissolution rate.

As used herein, the term “organic solvent” refers to a chemical substance used to dissolve materials within the system. The first organic solvent is utilized in the dissolution chamber to dissolve the polyester fraction of the textile material, while the second organic solvent is used to precipitate polyester. Selection of organic solvent depends on compatibility with the target materials and efficiency in the dissolution or precipitation process. Under exemplary operating conditions, the organic solvent operates within controlled temperatures and concentrations to maximize dissolution and precipitation outcomes.

As used herein, the term “suspension” or “dispersion” or “colloidal solution” refers to a mixture of undissolved material in solvent. The “suspension” or “dispersion” is subsequently processed in filter to separate solid from the solvent mixture. The stability and particle size distribution of “dispersion” or “colloidal solution” are managed for filtration and recovery of solid material and/or solvent.

As used herein, the term “filtering unit” refers to a device that separates solid materials from liquids. The filtering unit receives the suspension of undissolved material in the solvent and separates it into a liquid stream and a solid fraction. The pore size of the filter determines the size of particles that can pass through the filter, thus controlling the quality and purity of the separated materials. Filters with smaller pore sizes are used to capture finer particles, whereas larger pore sizes are suitable for initial filtration stages.

As used herein, the term “liquid stream” refers to the liquid output from the filtering unit, containing dissolved substance in the solvent. This stream is directed to various post processing stages. The polyester liquid stream can isolate the polyester fraction from the mixture, allowing for polyester subsequent recovery and reuse. The stream's composition and flow rate are managed for precipitation and separation in the following stages.

As used herein, the term “solid cotton fraction” refers to the solid residue of cotton fibers separated from the suspension in the first filtering unit. Separation and processing of the solid cotton fraction can maximize yield of reusable cotton fibers and minimize waste.

As used herein, the term “precipitation unit” refers to a component that facilitates the formation of solid particles from a solution. The precipitation unit receives the solution that comprises dissolved solute. The second solvent is mixed with the solution to generate a precipitate of solute. Precipitation enables recovery and purification of solute from the dissolved state and converting solute into a solid form. The precipitation unit may comprise features such as temperature-controlled chambers and baffles to optimize the precipitation process.

As used herein, the term “solid polyester” refers to the polyester material recovered in solid form after precipitation and filtration. The solid polyester is separated from the solvent mixture in the filter. This recovered polyester can be reused in manufacturing new textile products, blended textile, reducing the demand for virgin polyester and contributing to sustainability efforts.

As used herein, the term “cocktail” refers to the mixture of the solvents obtained after separating solid polyester in the second filter. This mixture is processed in the solvent recovery unit to separate and recycle the individual solvents.

As used herein, the term “solvent recovery unit” refers to a device that separates and recycles solvents from mixtures/cocktail. The solvent recovery unit receives the cocktail and separates the various solvents, thereby reducing waste and operational costs.

As used herein, the term “bioreactor” refers to a vessel designed for biological reactions, specifically enzymatic treatment. The bioreactor receives raw material (e.g., solid cotton fraction) and enables enzymatic treatment in a controlled environment in treatment medium, which comprises an enzyme, water and other components to facilitate the breakdown of cotton fibers. The bioreactor's conditions, such as temperature and enzyme concentration, are optimized for treatment and the production of various products.

As used herein, the term “treatment medium” refers to the mixture within the bioreactor that facilitates the enzymatic breakdown of raw product. The treatment medium comprises water, enzymes and other components necessary for the treatment process. The treatment medium's composition and conditions, such as pH and temperature, can be controlled to maximize the outcome of enzymatic treatment.

As used herein, the term “enzyme” refers to a biological catalyst used in the bioreactor to facilitate the treatment of cotton fibers. Enzymes accelerate chemical reactions, such as the breakdown of cellulose in cotton. The concentration and activity of the enzyme in the bioreactor are optimized for treatment and the production of high-quality products.

As used herein, the term “sugar recovery unit” refers to a device that removes sugar from the liquid fraction. The unit produces treatment medium that is recycled back to the bioreactor. Sugar recovery generates enzyme concentration and activity in the bioreactor. The removed sugar can be used for various purposes such as generation of biofuel, another metabolic byproduct etc.

As used herein, the term “self-cleaning mechanism” refers to a feature in the filtering units that enable removal of accumulated particles from the filter surface. This mechanism involves motor-driven brushes that sweep across the filter surface to maintain optimal filtration flow rates. The self-cleaning mechanism enables continuous operation and reduces maintenance requirements for the filtering units.

As used herein, the term “bleaching unit” refers to a device positioned upstream of the dissolution chamber that applies a bleaching agent to textile fragments to remove dye. The bleaching unit utilizes chemical spray nozzles, a mixing chamber and water jets. The bleaching process enhances the purity of the recovered fibers by removing unwanted dyes. As used herein, the chemical spray nozzles apply a bleaching agent to textile fragments. The nozzles enable the application of bleaching agent for dye removal.

As used herein, the term “bleaching agent” refers to a chemical substance used in the bleaching unit to remove dye from textile fragments. Selection of bleaching agent depends on effectiveness in removing specific dyes and compatibility with the textile fibers.

As used herein, the term “dissolves” refers to the process of causing a solid material/solute to solubilize into solvent to form solution. Selective dissolution process separates mixture of solutes, by control of dissolution conditions, such as temperature and solvent concentration.

Referring to FIG. 1, there is shown a block diagram of a system 100 to process textile waste 102, according to an embodiment of the present disclosure. The system (100) comprises a dissolution chamber (104), a first filtering unit (110), a precipitation unit (116), a first solvent recovery unit (128), a bioreactor (130), a third filter (136) and a sugar recovery unit (142). Each of these elements are interconnected through a series of valves, pumps and conduits that enable the controlled flow of materials.

In an embodiment, the dissolution chamber 104 receives textile waste 102. The waste fabric 102 can comprise post-consumer textiles such as discarded clothing, fabric scraps from garment manufacturing and used home textiles like bed linens and curtains. The waste fabric 102 includes polyester-cotton blends, which are utilized for the recycling process described herein.

By targeting commonly available textile waste 102, system 100 reduces landfill waste and promotes circular economy practices in the textile industry. The dissolution chamber 104 stores a first organic solvent 106. The first organic solvent 106 dissolves a polyester fraction of the received textile material to produce a suspension 108 of undissolved cotton in the first organic solvent 106. The dissolution chamber 104 enables the separation of polyester from cotton, required for recycling. By dissolving the polyester, the first organic solvent 106 facilitates the isolation of cotton, thereby improving recycling process. Furthermore, the dissolution chamber 104 can be constructed from materials (e.g., stainless steel) resistant to the first organic solvent 106, for longevity and reliability. For the dissolution chamber 104, the operational environment maintains a temperature range of 50° C. to 90° C. under atmospheric pressure.

In an embodiment, the first organic solvent 106 can be a glycol-based solvent such as dichloromethane, dimethylformamide, ethylene glycol or a glycol ether such as ethylene glycol monomethyl ether. The first organic solvent can be selected for its ability to dissolve polyester, while remaining inert towards cotton fibers. The dissolution chamber 104 may comprise a pressure release valve and a cooling jacket to maintain pressure fluctuations arise due to heating first organic solvent 106. The pressure release valve regulates any excess pressure build-up, for safe operation, while the cooling jacket helps to dissipate excess heat, maintaining the desired temperature range. Further, dissolution chamber 104 comprises an electric heating jacket to provide uniform heat distribution for dissolution of polyester, leading to a high-quality separation process.

In an embodiment, the dissolution chamber 104 employs a first organic solvent. The first organic solvent is a polar aprotic solvent selected from trichloroacetic acid (TCA), tetrahydrofuran (THF), N-methyl-2-pyrrolidone (NMP), acetone, hexafluoroisopropanol (HFIP), dichloromethane, and combination thereof. The dissolution chamber 104 maintains a dissolution temperature between 20-60° C. or 30-45° C., 35-37° C., 35-42° C. and like, a dissolution pressure of 35-55 kPa, 40-45 kPa, 40-50 kPa and the like, and a dissolution time ranging from 30-120 minutes, 45-60 minutes, 20-80 minutes, 90-120 minutes and the like. Optionally, combination of multiple solvents can be used as first solvent to dissolve polyester in varying ratios. For instance, a combination of tetrahydrofuran (THF) and dichloromethane in a ratio of 3:1 can be effective. Another combination could be N-methyl-2-pyrrolidone (NMP) and trichloroacetic acid (TCA) in a ratio of 2:1. Additionally, a mixture of acetone and hexafluoroisopropanol (HFIP) in equal parts can be utilized. Use of multiple solvents to dissolve polyester enabling more efficient dissolution of polyester, improve the dissolution rate, reducing the time required for the process. The synergistic mixture of solvents often together creates a more effective medium for breaking down polyester chains. Furthermore, the use of multiple solvents can improve the dissolution rate, reducing the time required for the process. Non-limiting examples of polyester based textile can be processed by system (100) are polyethylene terephthalate (PET), polytrimethylene terephthalate (PTT), polybutylene Terephthalate (PBT) and aramid.

In an embodiment, first filtering unit 110 receives the suspension 108 of undissolved cotton in the first organic solvent 106 and generates a polyester liquid stream 112 and a solid cotton fraction 114. The first filtering unit 110 enables the separation of the cotton fibers from the polyester solution, for recovering pure cotton. The filtering process segregates solid particles from the liquid to enable the purity of both the cotton and polyester outputs. Filters used within the first filtering unit 110 can be made of materials such as polypropylene or polytetrafluoroethylene (PTFE) due to their chemical resistance and mechanical strength. The first filtering unit 110 can maintain a pressure range of 1 to 5 bar and a filtration temperature between 25° C. and 60° C., which helps in separating the polyester liquid. Optionally, first filtering unit 110 can utilize a backwashing mechanism for preventing clogging and maintaining consistent filtration performance over extended periods.

In an embodiment, precipitation unit 116 receives the polyester liquid stream 112 and a second organic solvent 118 to generate a precipitated polyester dispersion 120. Precipitation unit 116 enables recovery of polyester from the liquid stream 112 by precipitating the polyester out of the polyester liquid stream 112. The precipitation enables recovery of reusable polyester material for waste fabric 102. The second organic solvent 118 can induce precipitation without reacting with the polyester. Exemplary second solvent 118 can be methanol or ethanol. The precipitation unit 116 operates at temperatures ranging from 10° C. to 40° C. and under atmospheric pressure to optimize the precipitation process. The precipitation unit 116 can be fabricated from glass-lined steel or high-density polyethylene for resistance to the solvents used and the mechanical stresses of the process. The precipitated polyester dispersion 120 produced by the precipitation unit 116 can then be further processed to recover pure polyester to provide a high-quality material for reuse in textile manufacturing. In an embodiment, a spraying mechanism can be employed to introduce the second organic solvent 118 into the polyester liquid stream 112 within the precipitation unit 116. The spraying mechanism allows for the second organic solvent 118 to be evenly distributed throughout the polyester liquid stream 112, enabling a consistent and thorough precipitation process. The spraying mechanism comprises atomizers to atomize the second organic solvent 118, creating fine droplets that mix readily with the polyester liquid stream 112.

In an embodiment, the second organic solvent is selected from methanol, ethanol, isopropanol, diethyl ether, hexane, cyclohexane, and combination thereof. Precipitation unit 116 maintains a precipitation temperature between 30-60° C., 25-55° C., 35-55° C., 40-50° C., 50-60° C., 55-75° C., and the like, a precipitation pressure of 40-65 kPa, 45-55 kPa, 35-55 kPa, 50-55 kPa, 55-60 kPa, 55 kPa and the like. and a precipitation time ranging from 20-500 minutes, 50-300 minutes, 100-650 minutes, 1-7 hours, 3-9 hours, and the like. The second organic solvent facilitates the precipitation process, enabling the effective recovery of dissolved polyester materials from dissolution chamber 104. Precipitation unit 116 includes enhanced separation and recovery of components.

In yet another embodiment, combination of solvents can be employed in various ratios to precipitate dissolved polyester. For an instance, combination of ethanol and diethyl ether in a ratio of 1:2 can be as effective as second solvent. Another combination could be isopropanol, cyclohexane and hexane in a ratio of 1:1:1. Additionally, methanol and cyclohexane in a ratio of 3:1 can be utilized. The use of combination of solvents for the precipitation of polyester improves selectivity for precipitating the desired polyester while minimizing impurities. By adjusting the solvent ratios, quality and yield of the precipitated polyester can be improved.

In an embodiment, the second filter (122) receives the precipitated polyester dispersion (120) to separate a solid polyester (124) from a cocktail (126) based on the first organic solvent (106) and the second organic solvent (118). The second filter (122) separates the solid polyester (124) by utilizing selective filtration techniques. The second filter (122) may comprise a microfiltration membrane or a nanofiltration membrane which allows passage of the cocktail (126) while retaining the solid polyester (124). The separation of the solid polyester (124) from the cocktail (126) facilitates the purification of polyester materials recovered from the textile waste (102). The second filter (122) operates at varying pressures to accommodate different viscosities of the precipitated polyester dispersion (120), thereby enhancing the adaptability of the system (100) to process various types of textile waste (102).

In an embodiment, solid polyester (124) undergoes a series of washing steps to remove residual solvent and impurities from the solid polyester (124). The washing process involves the introduction of a washing solution into the second filter (122). The washing solution may include deionized water, diluted organic solvents, or a combination thereof. The washing solution flows through the filtration membranes of the second filter (122), displacing and carrying away the residual solvents (used for dissolving or precipitating polyester) and impurities. Washing can be repeated multiple times to enable thorough removal of contaminants.

In an embodiment, the solid polyester (124) separated by the second filter (122) undergoes a bleaching process to remove any residual dye from the textile waste (102). The bleaching process involves the application of a bleaching agent, such as hydrogen peroxide or sodium hypochlorite, to the solid polyester (124). The bleaching agent reacts with the dye molecules, breaking them down and removing color from the polyester. After the bleaching process, the solid polyester (124) can be rinsed with water to remove any remaining bleaching agent and degraded dye residues.

In an embodiment, the second filter (122) incorporates a mechanism for the periodic removal of accumulated solid polyester (124) to maintain an optimal filtration rate. The mechanism comprises automated scraping. During operation, solid polyester (124) accumulates on the surface of the filtration membranes, reducing filtration. The automated system activates at pre-set intervals or when a specified pressure differential is detected across the filter. The mechanical scrapers gently remove the solid polyester (124) from the membrane surface, allowing it to be collected and discharged from the filtration unit.

In an embodiment, the textile waste processing system (100) employs a centrifugation step before the filtration process in the second filter (122) to expedite filtration. The centrifugation unit receives the precipitated polyester dispersion (120) and applies centrifugal force to separate the heavier solid polyester (124) from the lighter cocktail (126) based on the first organic solvent (106) and the second organic solvent (118). The separated solid polyester (124) is then fed into the second filter (122) for further purification. By pre-concentrating the solid polyester (124) through centrifugation, the filtration load on the second filter (122) is significantly reduced. This reduction in load enhances the filtration rate, allowing for quicker processing of the precipitated polyester dispersion (120). The centrifugation step also minimizes the risk of clogging and fouling of the filtration membranes within the second filter (122), which maintains optimal filtration and extends operational lifespan of filtration unit.

In an embodiment, a first solvent recovery unit (128) receives the cocktail (126) to separate the first organic solvent (106) and the second organic solvent (118), wherein the first organic solvent (106) is transferred to the dissolution chamber (104) and wherein the second organic solvent (118) is transferred to the precipitation unit (116). The first solvent recovery unit (128) operates by employing a series of separation techniques, such as distillation or solvent extraction, to recover the first organic solvent (106) and the second organic solvent (118) from the cocktail (126). The first solvent recovery unit (128) utilizes temperature and pressure variations to achieve selective separation of the solvents based on their boiling points and affinities. The first organic solvent (106) is redirected to the dissolution chamber (104) to dissolve further textile waste (102), while the second organic solvent (118) is sent to the precipitation unit (116) to facilitate the precipitation of polyester. The recovery of the solvents in the first solvent recovery unit (128) reduces the overall consumption of fresh solvents, thereby inexpensive and environmental sustainability of the system (100).

In an embodiment, the first solvent recovery unit (128) utilizes membrane separation technique to separate the first organic solvent (106) and the second organic solvent (118) from the cocktail (126). The first solvent recovery unit (128) utilizes membranes with selective permeability, allowing the passage of one solvent while retaining the other. The system (100) can use a supercritical fluid (for an instance, supercritical carbon dioxide) to selectively dissolve and extract one solvent from the cocktail (126). Additionally, the first solvent recovery unit (128) may implement a hybrid approach which involves combining multiple separation techniques. For instance, an initial membrane separation can be followed by distillation or adsorption to achieve a higher degree of solvent purity. By integrating various separation methods, the first solvent recovery unit (128) can handle a wider range of solvent mixtures and improve flexibility.

In an embodiment, a bioreactor (130) receives the solid cotton fraction (114), wherein the bioreactor (130) enables enzymatic treatment of the received cotton in a treatment medium (132) comprises an enzyme (e.g., cellulase, pectinase, hemicellulose, amylase, laccase, etc.), water, at a controlled temperature, an enzyme concentration for a pre-set time period to generate a colloidal solution of the cotton fibers. The bioreactor (130) facilitates the enzymatic breakdown of the solid cotton fraction (114) by providing optimal conditions for enzyme activity. The treatment medium (132) within the bioreactor (130) comprises specific enzymes, such as cellulase or pectinase, which target the cotton fibers and convert them into a colloidal solution having cotton fibers have dimensions. The bioreactor (130) maintains a controlled temperature, typically in the range of 30-60 degrees Celsius and an appropriate enzyme concentration. The duration of the enzymatic treatment process is predetermined based on the characteristics of the cotton fibers and the desired end-product quality. The enzymatic treatment in the bioreactor (130) produces a colloidal solution that can be further processed or utilized in various applications, such as textile manufacturing or bio-based products. The use of enzymes in the bioreactor (130) provides a sustainable alternative to chemical processes, reducing environmental impact and energy consumption. The type of enzyme used in the bioreactor (130) and the residual time of treatment may impact on the overall yield and fiber length of the digested cotton fibers. Different enzymes target specific components of cotton fibers, such as cellulose, pectin, or hemicellulose, resulting in varying degrees of treatment and fiber size. For instance, cellulase breaks down cellulose into smaller glucose units, producing finer fibers, whereas pectinase targets pectin, leading to partial degradation and larger fiber dimensions. The residual time of treatment, which refers to the duration the cotton fibers are exposed to the enzyme, also affects the extent of treatment. Shorter treatment times result in partial treatment, yielding longer fibers, while extended treatment times lead to more complete breakdown, producing shorter fibers. Balancing the type of enzyme and residual time impacts in achieving the desired fiber length and maximizing overall yield. The optimization of these parameters results in production of high-quality cotton fibers for specific applications.

In another embodiment, the bioreactor 130 utilizes a combination of multiple enzymes selected from cellulase, hemicellulase, pectinase, laccase, amylase, xylanase, protease, lipase and mannanase. The treatment medium (132) comprises a buffer (e.g., sodium acetate buffer, citrate buffer, phosphate buffer, Tris buffer, and MES buffer) to maintain pH level (e.g. 3-7.5, 6-6.5, 6.5-7, 7-7.5 and the like) during the enzymatic treatment of cotton. The bioreactor 130 operates at a temperature (e.g., 15-60° C., 35-45° C., 40-60° C., 15-60° C., etc.), a pressure ranging from 50 to 80 kPa, and treatment time from 15 minutes to 30 days. The combination of cellulase and hemicellulase enzymes in specified conditions enable breakdown of cellulose and hemicellulose present in the textile waste 102. Bioreactor 130 includes efficient conversion of textile waste 102 into fermentable sugars. During enzymatic treatment, the pH of treatment medium (132) may change due to the production of acidic or basic by-products. Enzymatic reactions often produce organic acids that can lower the pH, making the treatment medium (132) more acidic over time. Such a change in pH can affect the impact of the enzymes, as most enzymes have an optimal pH range in which they function most effectively. For instance, sodium acetate buffer can neutralize excess hydrogen ions (H+) produced during the treatment, preventing the pH from dropping too low. Similarly, phosphate buffer can counteract any pH fluctuations by providing either hydrogen ions (H+) or hydroxide ions (OH−) as needed to maintain a stable pH. By stabilizing the pH, the buffer enables that the enzymatic reactions continue at higher efficiency, allowing for consistent and effective treatment of cotton. Treatment time, enzyme type, and enzyme concentration can be optimized to control fiber length. Prolonged treatment time can lead to excessive fiber breakage, reducing fiber length or generation of various metabolite (e.g., sugar) to reduce fiber yield. Optimal treatment time ranges from 15 minutes to 30 days. Enzyme concentration also affects the extent of treatment, higher concentrations can accelerate the process but may risk fiber damage. Cotton fibers produce after treatment may have fiber length in range 5 to 50 mm, 15-20 mm, 20-25 mm, 25-30 mm, 30-35 mm, and 35-40 mm.

In an embodiment, before enzymatic treatment, the solid cotton fraction (114) is subjected to a washing process to remove any residual organic solvents or adsorbed polyester. The washing process involves multiple stages where the solid cotton fraction (114) is rinsed with water or an aqueous solution, for thorough removal of contaminants. The washing process may also comprise the use of surfactants to enhance the removal of organic solvents. The washed cotton fibers are then dried to remove any remaining moisture or organic solvent, resulting in clean and purified fibers ready for further processing or utilization.

In an embodiment, various additives can be introduced into the treatment medium (132) to improve the efficiency of the enzyme during the treatment process. Such additives may comprise metal ions like calcium or magnesium, which act as cofactors and enhance enzyme stability and activity. Additionally, buffer solutions are used to maintain the optimal pH for enzyme activity. Surfactants and detergents may also be added to increase the accessibility of the enzyme to the cotton fibers by reducing surface tension.

In an embodiment, immobilized enzymes can be employed within the bioreactor (130) to retain enzyme concentration and improve the treatment process. Immobilized enzymes are enzymes that are fixed onto a solid support, such as beads or membranes, allowing them to be reused and maintained within the reaction environment. The use of immobilized enzymes prevents enzyme loss during the treatment process and provides consistent enzymatic activity over extended periods.

In an embodiment, the enzyme used within the bioreactor (130) can be derived from a bacterial strain (e.g., Thermobifida fusca) known for producing robust enzymes capable of withstanding harsh conditions such as higher temperatures.

In an embodiment, third filter (136) receives the colloidal solution of the cotton fibers (102) and separates the cotton fibers (138) and a liquid fraction (140), which comprises the enzyme and sugar can be single carbon sugar to 6 carbon sugar as well as a variety of alcohol derivatives. The third filter (136) allows the colloidal solution to pass through a filtering medium, which retains the cotton fibers (138) while permitting the liquid fraction (140) to pass through. The cotton fibers (138) are then collected for further processing or disposal. The liquid fraction (140), which contains the enzyme and sugar, is directed to subsequent processing units. The separation achieved by the third filter (136) allows: processing of liquid fractions, free from solid contaminants to reduce load on downstream units, leading to increased operational processing and reduced maintenance requirements.

In an embodiment, a sugar recovery unit (142) receives the liquid fraction (140) from the third filter (136) and removes the sugar from the liquid fraction (140) to produce treatment medium (132) that is transferred to the bioreactor (130). The sugar recovery unit (142) employs techniques such as crystallization, adsorption, or membrane filtration to isolate the sugar from treatment medium (132). The purified treatment medium (132), is then transferred to the bioreactor (130) for further biochemical reactions. The removal of sugar from the liquid fraction (140) by the sugar recovery unit (142) maintains the activity and stability of the enzyme in subsequent processes. By removing the sugar, the sugar recovery unit (142) retains catalytic property of enzyme, thereby improving the overall yield of the bioreactor (130). This step also allows for the recovery of valuable sugar, which can be utilized in other industrial processes, adding to the economic viability of the system.

In an embodiment, a control unit facilitates the transfer of various materials into and out from multiple elements within the system (100) to process textile waste (102). The control unit enables control of the transfer of materials into and out of various elements. The control unit regulates the introduction of textile waste (102) and the first organic solvent (106) into the dissolution chamber (104). The control unit monitors the concentration and flow rate of the first organic solvent (106) for dissolution of the polyester fraction. Upon achieving the desired dissolution, the control unit controls flow of the suspension (108) of undissolved cotton in the first organic solvent (106) to the first filtering unit (110) through automated valves and pumps. Further, the control unit manages transfer of the polyester liquid stream (112) from the first filtering unit (110) to the precipitation unit (116). The control unit manages the timing and volume of the polyester liquid stream (112) to optimize the precipitation process. Concurrently, the control unit directs the solid cotton fraction (114) from the first filtering unit (110) to the bioreactor (130), adjusting flow rates and manages that the solid cotton fraction (114) is adequately prepared for enzymatic treatment. Moreover, the control unit regulates the introduction of the second organic solvent (118) to the polyester liquid stream (112) within the precipitation unit (116). The control unit maintains the appropriate solvent ratios and mixing times to generate a uniform precipitated polyester dispersion (120). The control unit transfers the precipitated polyester dispersion (120) to the second filter (122), thereby transfer is smooth. The control unit directs the separated solid polyester (124) from the second filter (122) for collection and manages the transfer of the cocktail (126) of the first and second organic solvents to the first solvent recovery unit (128). Within the first solvent recovery unit (128), the control unit oversees the separation and recycling processes, ensuring that the first organic solvent (106) is recovered and transferred back to the dissolution chamber (104), while the second organic solvent (118) is redirected to the precipitation unit (116). The control unit maintains optimal conditions within the bioreactor (130) for the enzymatic treatment of the solid cotton fraction (114). The control unit regulates temperature, enzyme concentration and treatment time. The control unit facilitates the transfer of the resulting colloidal solution of cotton fibers to the third filter (136) once the treatment process is complete. The control unit regulates the operation of the third filter (136) to separate the cotton fibers (138) from the liquid fraction (140). The control unit monitors the filtration process for separation and subsequently, directs the liquid fraction (140) to the sugar recovery unit (142). Within the sugar recovery unit (142), the control unit oversees the removal of sugar from the liquid fraction (140), producing treatment medium (132). Finally, the control unit transfers the purified treatment medium (132) back to the bioreactor (130), to allow continuous operation of the textile waste processing system (100). The control mechanism utilizes valves, pumps and conduits to regulate flow rates and maintain process conditions.

In an embodiment, each of the first filtering unit (110) and the third filter (136) comprises a self-cleaning mechanism. Each self-cleaning mechanism comprises multiple motor-driven brushes configured to sweep across a filter surface to dislodge particles accumulated on the filter surface. The motor-driven brushes operate in a synchronized manner to enable continuous cleaning of the filter surfaces, thereby preventing clogging and maintaining optimal filtration performance. The brushes cover the entire filter surface and movement is controlled by the control unit to enable timely activation and deactivation. The self-cleaning mechanism enhances the longevity of the filters by minimizing downtime for manual cleaning and reducing the risk of filter damage due to particle buildup. The continuous operation of the motor-driven brushes maintains cleanliness of filters.

In an embodiment, each of the first filtering unit (110) and the third filter (136) comprises a plurality of vibrators attached to the exterior of the filter housing. Each vibrator generates vibrations at specific intervals to maintain a filtration flow rate. Each vibrator enables vibrations to be evenly distributed across the filter housing, preventing the accumulation of particles on the filter surface and maintaining flow rate through the filters. The control unit regulates the frequency and intensity of the vibrations to match the filtration requirements and the type of material being processed. The use of vibrators helps to dislodge particles that may adhere to the filter surface, thereby reducing the risk of clogging. This mechanism enhances the reliability of the filtration units, contributing to the overall performance of the textile waste processing system (100).

In an embodiment, the bioreactor (130) comprises immobilized enzyme beads. The immobilized enzyme beads provide a stable and reusable medium for the enzymatic treatment of the solid cotton fraction (114). The beads are composed of a porous material that allows enzymes to be securely attached while maintaining their catalytic activity. The immobilization of enzymes on beads prevents enzyme loss and enables the continuous operation of the bioreactor (130) without the need for frequent enzyme replenishment. The beads are suspended in the treatment medium (132) within the bioreactor (130) for breakdown of cotton fibers into a colloidal solution. The use of immobilized enzyme beads enhances the stability of the enzymatic treatment process, resulting in higher yields and reduced operational costs. Additionally, the beads can be easily separated from the treatment medium (132) for cleaning and reuse, further improving the sustainability of the textile waste processing system (100).

In an embodiment, the precipitation unit (116) comprises a temperature-controlled chamber. The temperature-controlled chamber can be a double-walled structure with circulating fluid between the double walls to maintain a stable temperature during the precipitation process. The circulating fluid, which can be either heated or cooled, is regulated by the system's control unit to achieve desired temperature conditions within the chamber. The precise temperature control provided by the double-walled chamber enables optimal conditions for the precipitation of polyester from the polyester liquid stream (112). Maintaining a stable temperature achieves uniform particle size and prevents agglomeration during precipitation. The temperature-controlled chamber amplifies precipitation process, resulting in a high-quality precipitated polyester dispersion (120).

In an embodiment, the sugar recovery unit (142) comprises a sugar affinity column filled with plurality of affinity beads to selectively bind sugar while allowing enzymes to pass through. The affinity beads with specific binding sites that have a high affinity for sugar molecules. When the liquid fraction (140) containing both enzymes and sugar passes through the sugar recovery unit (142), the affinity beads (of sugar affinity column) capture the sugar due to their selective binding properties for separation of sugar from treatment medium (132). The treatment medium (132), now free from sugar, is collected and transferred back to the bioreactor (130). The use of affinity beads in the sugar recovery unit (142) removes sugar, maintains enzyme activity and prevents inhibition during subsequent reactions.

In another embodiment, the second filter (122) further comprises a centrifuge mechanism to separate the solid polyester (124) from the cocktail (126). The centrifuge mechanism employs centrifugal force to accelerate the separation process. When the precipitated polyester dispersion (120) enters the second filter (122), the centrifuge spins at high speeds, causing the solid polyester (124) to move outward due to higher density, while the cocktail (126) remains closer to the center. By utilizing separation technique, solid polyester (124) is isolated from the cocktail (126), which contains the first organic solvent (106) and the second organic solvent (118).

In an embodiment, the bioreactor (130) comprises an automated pH control unit to maintain optimal conditions for enzymatic treatment. The pH control unit includes sensors and actuators that continuously monitor and adjust the pH level of the treatment medium (132). Maintaining an optimal pH provides activity and stability of the enzymes involved in the treatment process. The automated pH control unit enables that the pH remains within a narrow range, which enhances enzyme performance and maximizes the treatment process. By preventing fluctuations in pH, the system avoids conditions that could denature the enzymes or slow down the reaction rates.

In another embodiment, the precipitation unit (116) comprises a series of baffles to facilitate the separation of precipitated polyester. The baffles can create a controlled flow pattern for the liquid stream. As the polyester liquid stream (112) mixes with the second organic solvent (118), the precipitated polyester dispersion (120) forms and moves through the baffles. The baffles slow down the flow and provide surfaces for the precipitated polyester to coalesce and settle. The series of baffles improves the separation by promoting the aggregation of polyester particles, making it easier to isolate the solid polyester (124) in the subsequent second filter (122). The incorporation of baffles in the precipitation unit (116) amplifies the overall separation process and contributes to the quality and purity of the recovered polyester.

In an embodiment, the bioreactor (130) includes a temperature-controlled jacket to maintain temperature throughout the enzymatic treatment process. The temperature-controlled comprises a heating element and a cooling element. The temperature-controlled jacket allows for temperature regulation by circulating a temperature-controlled fluid around the bioreactor (130). Temperature fluctuations can affect reaction rates and enzyme stability. The temperature-controlled jacket enables that the treatment medium (132) remains at the ideal temperature for enzymatic treatment, thereby improves bioreactor (130) functioning.

In another embodiment, the sugar recovery unit (142) comprises a membrane filtration system to concentrate the treatment medium (132) before recycling into the bioreactor (130). The membrane filtration system uses semi-permeable membranes to separate treatment medium (132) from other components based on molecular size. As the liquid fraction (140) passes through the membrane filtration system, water and small molecules, such as sugar, are removed, concentrating treatment medium (132). This concentrated treatment medium (132) is then transferred back to the bioreactor (130). The use of a membrane filtration system in the sugar recovery unit (142) allows better utilization of enzyme recovery and reduces the need for fresh enzyme inputs, thereby lowering operational costs and improving the sustainability of the system (100).

In an embodiment, the system (100) further comprises a shredder unit positioned upstream of the dissolution chamber (104). The shredder unit comprises a conveyor belt to uniformly feed the textile waste (102) to a series of rotating blades. The conveyor belt enables controlled movement of the textile waste (102) towards the rotating blades, which shred the textile waste (102) into smaller textile fragments. This shredding process reduces the size of the textile waste (102), facilitating easier handling and processing in subsequent stages of the system. Additionally, the shredder unit comprises a dust extraction unit that captures and removes airborne particles generated during the shredding process. The dust extraction unit maintains a clean working environment and prevents the accumulation of dust that could potentially hinder the operation of the shredder unit and other downstream components. The inclusion of the shredder unit enables that the textile waste (102) is uniformly processed into manageable fragments, increasing the dissolution process in the dissolution chamber (104) and reducing the risk of blockages and mechanical issues within the system (100).

In an embodiment, the system (100) further comprises a bleaching unit positioned upstream of the dissolution chamber (104). The bleaching unit comprises a series of chemical spray nozzles that apply a bleaching agent to the textile fragments to remove dye. The chemical spray nozzles deliver even application of the bleaching agent for thorough coverage of the textile fragments. The bleaching unit or dye removal unit also includes a mixing chamber that enables uniform application of the bleaching agent on the textile fibers. The mixing chamber evenly distributes the bleaching agent across all the textile fragments, facilitating dye removal. Additionally, the bleaching unit comprises multiple water jets that spray water to remove residual bleaching agent and remove dye from the surface of the textile fragments. The water jets cater washing away, any remaining bleaching agent and dissolved dyes, leaving the textile fragments clean and ready for further processing in the dissolution chamber (104).

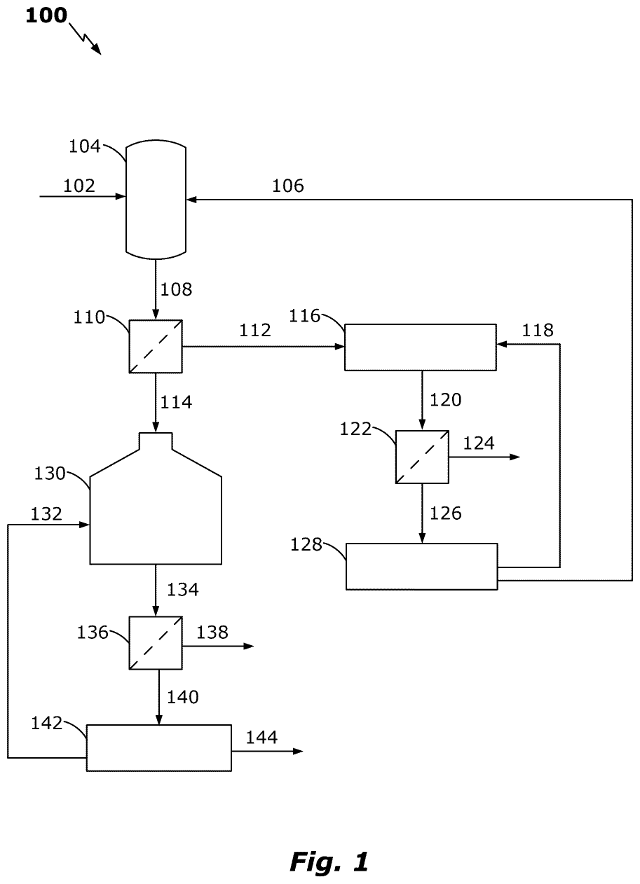

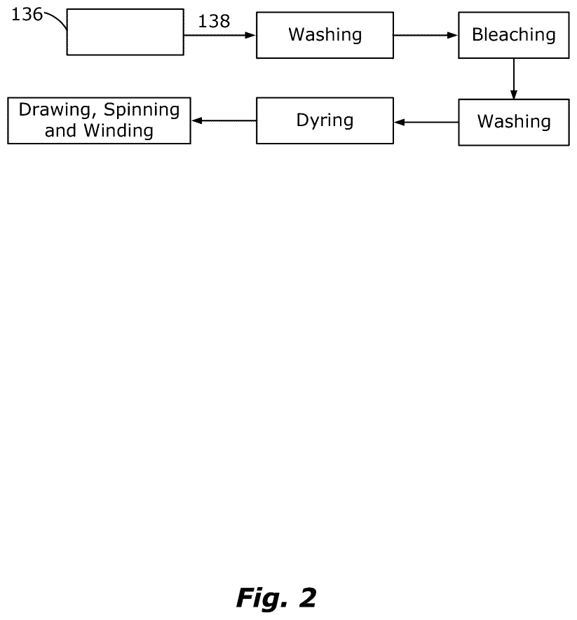

Referring to FIG. 2, there is shown a flow diagram illustrating processing of cotton fibers (138) subsequent to passage through the third filter (136), in accordance with an embodiment of the present disclosure. As shown, the cotton fibers (138) pass through the third filter (136) and are subsequently washed to remove dirt, debris, and other impurities. The washing process enables the removal of contaminants, thereby increasing quality of fibers. Thereafter, the cotton fibers (138) undergo a bleaching process. The process removes visually unappealing and non-uniform colors, thus providing a uniform appearance to the fibers. The bleaching process enhances the aesthetic quality of the cotton fibers and prepares them for further processing. Subsequent to the the cotton fibers (138) are subjected to a second washing to remove any residual bleach and other chemicals that may remain on the fibers. Thereafter, the cotton fibers (138) are dried. The drying process removes any remaining moisture from the fibers, making them suitable for subsequent processing steps. The dried cotton fibers (138) are then drawn, spun, or wound, depending on the desired end product. These processes convert the fibers into one continuous strand or yarn, spin together to form multifilament yarn, thread, which can be used in the manufacturing of textiles. The system (100) enables that the cotton fibers are processed in a manner that results in clean and uniformly colored for use in textiles to enable improved aesthetic quality for further textile manufacturing processes.

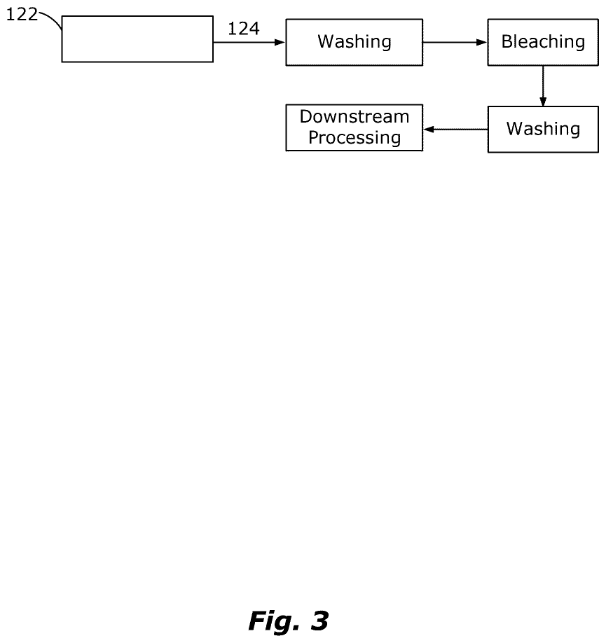

Referring to FIG. 3, there is shown a flow diagram illustrating processing of solid polyester (124) subsequent to passage through the second filter (122), in accordance with an embodiment of the present disclosure. Subsequent to passage of the solid polyester (124) through the second filter (122), the solid polyester is subjected to a washing process to eliminate dirt, debris, and other impurities. The washing process enables the purification of the polyester fibers, preparing them for further treatment. Subsequent to the washing, the solid polyester is subjected to a process. The bleaching process removes visually unappealing and non-uniform colors, resulting in a consistent and uniform appearance of the polyester fibers. This process is particularly important for maintaining the aesthetic quality of polyester. Thereafter, the solid polyester is washed again to remove any residual bleach and other chemicals. Subsequently, the solid is subjected to further downstream processing, such as, processing using various methods for transforming the treated polyester fibers into final products. For example, the downstream processing includes manufacturing different types of items such as clothing, home textiles, industrial fabrics, and other textile products. The treated polyester can be woven into fabrics, knitted into garments, or used in non-woven applications such as geotextiles and medical textiles. Such a stage is essential for converting the processed fibers into commercially viable and consumer-ready products.

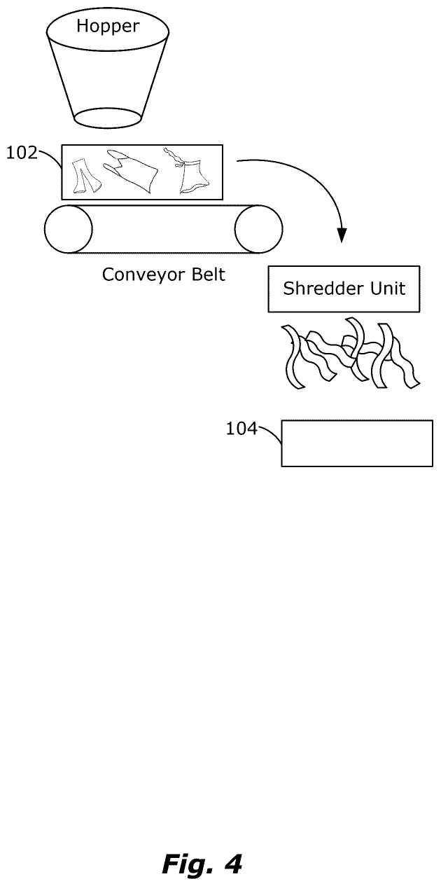

Referring to FIG. 4, there is schematically illustrated presence of an additional step in the processing of textile waste (102), in accordance with an embodiment of the present disclosure. As shown, the textile waste (102) is deposited onto a conveyor belt form a hopper. The hopper facilitates the controlled and smooth placement of the textile waste onto the conveyor belt, ensuring that the material is evenly distributed and preventing clogging or interruptions in the processing flow. The textile waste may contain unrequired material, knots, and clumps, which are addressed during the subsequent processing steps. Subsequently, the material is directed through a shredder unit. The shredder unit shreds the textile waste into uniform pieces. Such a shredding process reduces size of the textile waste to ease recycling process. The shredded pieces are thereafter deposited into the dissolution chamber (104). Additionally, the textile pieces may contain metal or metal-attached pieces that need to be removed, as such metals can interfere with downstream processing. The removal of these metals can prevent contamination and interaction with various solvent and/or enzyme and or block/damage filter surface.

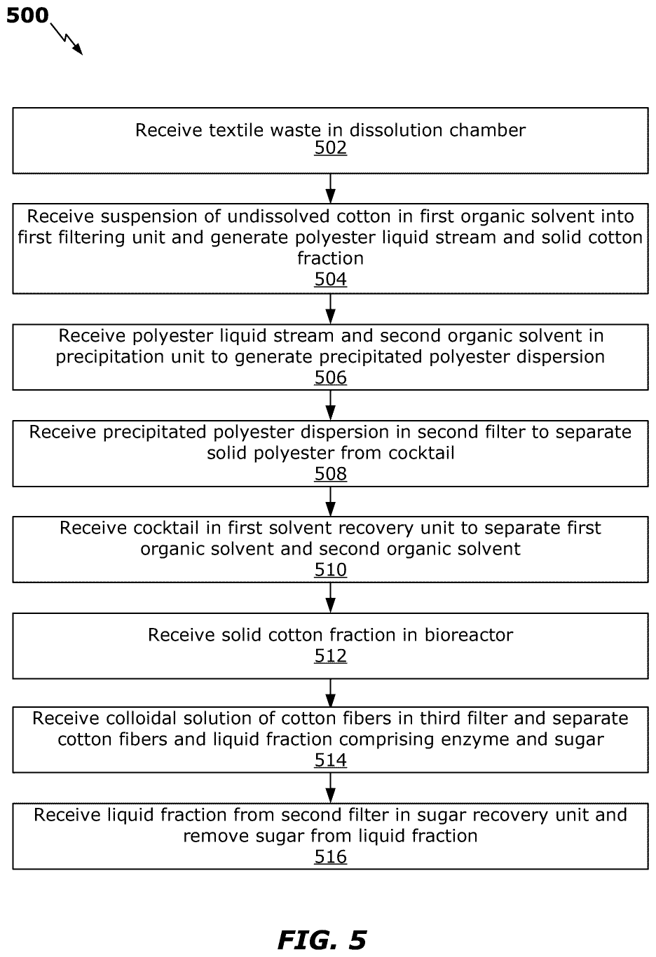

Referring to FIG. 5, there is shown a flowchart 500 illustrating a method of processing textile waste, in accordance with an embodiment of the present disclosure. At step 502, the textile waste is received in a dissolution chamber. The dissolution chamber stores a first organic solvent that dissolves a polyester fraction of the received textile material to produce a suspension of undissolved cotton in the first organic solvent. At step 504, the suspension of undissolved cotton in the first organic solvent is received into a first filtering unit and a polyester liquid stream and a solid cotton fraction are generated. At step 506, the polyester liquid stream and a second organic solvent are received in a precipitation unit to generate a precipitated polyester dispersion. At step 508, the precipitated polyester dispersion is received in a second filter to separate a solid polyester from a cocktail based on the first organic solvent and the second organic solvent. At step 510, the cocktail is received in a first solvent recovery unit to separate the first organic solvent and the second organic solvent. The first organic solvent is transferred to the dissolution chamber and the second organic is transferred to the precipitation unit. At step 510, the solid cotton fraction is received in a bioreactor. The bioreactor enables an enzymatic treatment of the received cotton in a treatment medium comprising at least one enzyme, water and buffer at a controlled temperature between 15° C. and 60° C., at a pre-set pressure ranging from 50 to 80 kPa, an enzyme concentration for a pre-set time period from 15 minutes to 30 days to generate a colloidal solution of the cotton fibers. At step 512, the colloidal solution of the cotton fibers is received in a third filter and the cotton fibers are separated from a liquid fraction comprising the enzyme and sugar. At step 514, the liquid fraction is received from the second filter in a sugar recovery unit and the sugar is removed from the liquid fraction to produce treatment medium that is transferred to the bioreactor.

Operations in accordance with a variety of aspects of the disclosure described above would not have to be performed in the precise order described. Rather, various steps can be handled in reverse order or simultaneously or not at all.

While several implementations have been described and illustrated herein, a variety of other means and/or structures for performing the function and/or obtaining the results and/or one or more of the advantages described herein may be utilized, and each of such variations and/or modifications is deemed to be within the scope of the implementations described herein. More generally, all parameters, dimensions, materials, and configurations described herein are meant to be exemplary and that the actual parameters, dimensions, materials, and/or configurations will depend upon the specific application or applications for which the teachings is/are used. Those skilled in the art will recognize or be able to ascertain using no more than routine experimentation, many equivalents to the specific implementations described herein. It is, therefore, to be understood that the foregoing implementations are presented by way of example only and that, within the scope of the appended claims and equivalents thereto, implementations may be practiced otherwise than as specifically described and claimed. Implementations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A system to process textile waste, wherein the system comprises:
a dissolution chamber receives the textile waste, wherein the dissolution chamber stores a first organic solvent that dissolves a polyester fraction of the received textile material to produce a suspension of undissolved cotton in the first organic solvent;
a first filtering unit receives the suspension of undissolved cotton in the first organic solvent and generates a polyester liquid stream and a solid cotton fraction;
a precipitation unit receives the polyester liquid stream and a second organic solvent to generate a precipitated polyester dispersion;
a second filter receives the precipitated polyester dispersion to separate a solid polyester from a cocktail based on the first organic solvent and the second organic solvent;
a first solvent recovery unit receives the cocktail to separate the first organic solvent and the second organic solvent, wherein the first organic solvent is transferred to the dissolution chamber and wherein the second organic is transferred to the precipitation unit;
a bioreactor receives the solid cotton fraction, wherein the bioreactor enables an enzymatic treatment of the received cotton in a treatment medium comprising at least one enzyme, water and buffer at a controlled temperature between 15° C. and 60° C., at a pre-set pressure ranging from 50 to 80 kPa, an enzyme concentration for a pre-set time period from 15 minutes to 30 days to generate a colloidal solution of the cotton fibers;
a third filter receives the colloidal solution of the cotton fibers and separates the cotton fibers, and a liquid fraction comprises the enzyme and sugar; and
a sugar recovery unit receives the liquid fraction from the second filter and removes the sugar from the liquid fraction to produce the treatment medium that is transferred to the bioreactor.

2. The system of claim 1, wherein the bioreactor comprises the immobilized enzyme beads.

3. The system of claim 1, wherein the precipitation unit comprises a double-walled chamber to receive a circulating fluid within the walls to maintain a thermal parameter during precipitation of the polyester.

4. The system of claim 1, wherein the sugar recovery unit comprises a sugar affinity column to selectively bind sugar while allowing the enzyme to pass therethrough.

5. The system of claim 1, wherein the second filter further comprises a centrifuge mechanism to separate the solid polyester from the cocktail based on the second organic solvent.

6. The system of claim 1, wherein the bioreactor comprises an automated pH control unit to maintain an optimal pH conditions during the enzymatic treatment.

7. The system of claim 1, wherein the precipitation unit comprises the baffles to facilitate separation of the precipitated polyester.

8. The system of claim 1, wherein the bioreactor comprises a temperature-controlled jacket to maintain a controlled temperature throughout the enzymatic treatment.

9. The system of claim 1, wherein the sugar recovery unit comprises a membrane filtration system to concentrate the treatment medium, before recycling to the bioreactor.

10. The system of claim 1, further comprises a shredder unit positioned at an upstream of the dissolution chamber, wherein the shredder unit comprises:
a conveyor belt to uniformly feed the textile waste to the blades to shred the feed waste textile into the textile fragments; and
a dust extraction unit to capture and remove the airborne particles.

11. The system of claim 1, further comprises a bleaching unit positioned upstream of the dissolution chamber, wherein the bleaching unit comprises:
the chemical spray nozzles to apply a bleaching agent to the textile waste to remove a dye; and
a mixing chamber to enable application of the bleaching agent on the textile waste.

12. The system of claim 1, wherein the first organic solvent is a polar aprotic solvent selected from trichloroacetic acid (TCA), Tetrahydrofuran (THF), N-Methyl-2-pyrrolidone (NMP), acetone, hexafluoroisopropanol (HFIP) dichloromethane, and combination thereof, wherein the dissolution chamber maintains a dissolution temperature between 20° C. and 60° C., a dissolution pressure of 55 kPa, and a dissolution time ranging from 30 minutes to 2 hours.

13. The system of claim 1, wherein the second organic solvent is selected from methanol, ethanol, isopropanol, diethyl ether, hexane, cyclohexane and combination thereof, wherein the precipitation unit maintains a precipitation temperature between 30° C. and 60° C., a precipitation pressure of 55 kPa, and a precipitation time ranging from 20 minutes to 8 hours.

14. A method of processing textile waste, wherein the method comprises:
receiving the textile waste in a dissolution chamber, wherein the dissolution chamber stores a first organic solvent that dissolves a polyester fraction of the received textile material to produce a suspension of undissolved cotton in the first organic solvent;
receiving the suspension of undissolved cotton in the first organic solvent into a first filtering unit and generating a polyester liquid stream and a solid cotton fraction;
receiving the polyester liquid stream and a second organic solvent in a precipitation unit to generate a precipitated polyester dispersion;
receiving the precipitated polyester dispersion in a second filter to separate a solid polyester from a cocktail based on the first organic solvent and the second organic solvent;
receiving the cocktail in a first solvent recovery unit to separate the first organic solvent and the second organic solvent, wherein the first organic solvent is transferred to the dissolution chamber and wherein the second organic is transferred to the precipitation unit;
receiving the solid cotton fraction in a bioreactor, wherein the bioreactor enables an enzymatic treatment of the received cotton in a treatment medium comprising at least one enzyme, water and buffer at a controlled temperature between 15° C. and 60° C., at a pre-set pressure ranging from 50 to 80 kPa, an enzyme concentration for a pre-set time period from 15 minutes to 30 days to generate a colloidal solution of the cotton fibers
receiving the colloidal solution of the cotton fibers in a third filter and separating the cotton fibers and a liquid fraction comprising the enzyme and sugar; and
receiving the liquid fraction from the second filter in a sugar recovery unit and removing the sugar from the liquid fraction to produce the treatment medium that is transferred to the bioreactor.

15. The method of claim 14, wherein the method comprises receiving a circulating fluid within a precipitation unit comprising a double-walled chamber to maintain a thermal parameter during precipitation of the polyester.

16. The method of claim 14, wherein the method comprises separating the solid polyester from the cocktail based on the second organic solvent in a centrifuge mechanism.

17. The method of claim 14, wherein the method comprises maintaining optimal pH conditions during the enzymatic treatment using automated pH control unit and wherein the bioreactor comprises the automated pH control unit.

18. The method of claim 14, wherein the method comprises maintaining a controlled temperature throughout the enzymatic treatment using a temperature-controlled jacket and wherein the bioreactor comprises the temperature-controlled jacket.

19. The method of claim 14, wherein the method comprises concentrating the treatment medium using a membrane filtration system, before recycling to the bioreactor and wherein the sugar recovery unit comprises the membrane filtration system.
